(19) United States
(12) Patent Application Publication
(10) Pub. No.: US 2018/0071340 A1
(43) Pub. Date: Mar. 15, 2018

(54) COMPOSITIONS AND METHODS OF TREATING MULTIPLE MYELOMA

(71) Applicants: Dana-Farber Cancer Institute, Inc., Boston, MA (US); Beth Israel Deaconess Medical Center, Boston, MA (US)

(72) Inventors: David AVIGAN, Sharon, MA (US); Jacalyn ROSENBLATT, Newton, MA (US); Donald KUFE, Wellesley, MA (US)

(21) Appl. No.: 15/563,164

(22) PCT Filed: Mar. 30, 2015
(86) PCT No.: PCT/US2016/024982

§ 371 Date: Sep. 29, 2017

Related U.S. Application Data
(60) Provisional application No. 62/140,330, filed on Mar. 30, 2015.

(51) Int. Cl. A61K 35/15; A61K 38/19; A61K 45/06

(52) U.S. Cl. CPC A61K 35/15; A61K 45/06; A61K 38/193

(57) ABSTRACT

The present invention provides compositions and methods for treating multiple myeloma.

### GOVERNMENT INTEREST

This invention was made with government support under P50 CA100707 awarded by the National Cancer Institute. The government has certain rights in the invention.

### FIELD OF THE INVENTION

The present invention relates generally to cellular immunology and more particularly to and methods for treating multiple myeloma (MM).

### BACKGROUND OF THE INVENTION

Multiple myeloma (MM) is a hematological malignancy associated with the increased presence of plasma cells. In the year 2000, approximately 13,700 people were diagnosed with multiple myeloma and it accounted for 2% of all cancer deaths. The clinical presentation is variable and may include bone pain, anemia, hypercalcemia and renal insufficiency. Prognostic factors include the plasma cell morphologic characteristics, serum beta-2 microglobulin levels, the plasma cell labeling index, and cytogenetics.

Multiple myeloma often responds initially to chemotherapy but long term disease control is elusive due to the emergence of chemotherapy resistant disease. As initial therapy, melphalan and prednisone is well tolerated and results in response rates of 50-60%. Prospective randomized studies have not consistently demonstrated improved outcomes with more intensive combination chemotherapy regimens. Use of vincristine, doxorubicin, and decadron is associated with high response rates, but no improvement in duration of response. Initial therapy with high dose decadron alone results in response rates only 15% less than that seen with VAD and similar to melphalan and prednisone.

Despite significant advancement in the treatment of myeloma, patients will ultimately experience disease progression due to the emergence of chemotherapy resistant disease. Although high dose chemotherapy with autologous stem cell rescue has been associated with improved disease free and overall survival, curative outcomes remain elusive. The potential susceptibility of multiple myeloma to immune based therapy has been demonstrated in the setting of allogeneic transplantation. Graft versus myeloma effect is thought to be responsible for the decreased risk of relapse and potential curative outcomes seen. Donor lymphocyte infusions given to patients who have experienced disease progression following transplantation have been associated with complete and partial responses. However, patients commonly develop graft versus host disease and associated complications due to the lack of tumor specificity of alloreactive T cells. Thus a need exists for MM specific immunotherapy.

### SUMMARY OF THE INVENTION

The invention features methods treating multiple myeloma in a patient by administering to the patient within 4 weeks of hematopoietic recovery following an autologous stem cell transplant a composition containing a population of autologous dendritic cell/multiple myeloma cell fusions (DC/MM fusions). The composition comprises about 1×10⁵ to 1×10⁶ DC/MM cell fusions. The patient receives one dose of DC/MM fusions prior to the autologous stem cell transplant. The composition is administered at four week intervals. The subject receives at least two doses of the composition.

In various aspects the method further includes administering GM-CSF. The GM-CSF is administered daily for 3 days. The GM-CSF is administered at a dose of 100 ug. The GM-CSF is administered at each dose of said DC/MM cell fusions.

In other aspects the method further includes administering to the subject a checkpoint inhibitor. The checkpoint inhibitor is administered one week after the DC/MM fusions. The checkpoint inhibitor is a PD1, PDL1, PDL2, TIM3, LAG3 inhibitor. Preferably, the checkpoint inhibitor is a PD1, PDL1, TIM3, LAG3 antibody.

In other aspects the method further includes administering to the subject an agent that target regulatory T cells

In a further aspect, the method further includes administering to the subject an immunomodulatory agent. The immunomodulatory agent is lenalidomide or pomalinomide or apremilast.

In yet another aspect, the method further includes administering to the subject a TLR agonist, CPG ODN, polyIC, or tetanus toxoid.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### DETAILED DESCRIPTION OF THE INVENTION

The invention features immune system-stimulating compositions that contain cells formed by fusion between autologous dendritic cells (DCs) and tumor cells. Specifically, the invention provides cell fusion of autologous DCs and multiple myeloma (MM) cells obtained from a subject that has MM. Also provide are methods of treating MM by administering to a patient whom has undergone an autologous stem cell transplant the autologous cell fusions according to the invention.

DCs can be obtained from bone marrow cultures, peripheral blood, spleen, or any other appropriate tissue of a mammal using protocols known in the art. Bone marrow contains DC progenitors, which, upon treatment with cytokines, such as granulocyte-macrophage colony-stimulating factor (“GM-CSF”) and interleukin 4 (“IL-4”), proliferate and differentiate into DCs. Tumor necrosis cell factor (TNF) is optionally used in conjunction with GM-CSF and/or IL-4 to promote maturation of DCs. DCs obtained from bone marrow are relatively immature (as compared to, for instance, spleen DCs). GM-CSF/IL-4 stimulated DC express MHC class I and class II molecules, B7-1, B7-2, ICAM, CD40 and variable levels of CD83. These immature DCs are more amenable to fusion (or antigen uptake) than the more mature DCs found in spleen, whereas more mature DCs are relatively more effective antigen presenting cells. Peripheral blood also contains relatively immature DCs or DC progenitors, which can propagate and differentiate in the presence of appropriate cytokines such as GM-CSF and-which can also be used in fusion.

Preferably, the DCs are obtained from peripheral blood.

The DCs must have sufficient viability prior to fusion. The viability of the DCs is at least 70%, at least 75%, at least 80% or greater.

Prior to fusion the population of the DCs are free of components used during the production, e.g., cell culture components and substantially free of mycoplasm, endotoxin, and microbial contamination. Preferably, the population of DCs has less than 10, 5, 3, 2, or 1 CFU/swab. Most preferably the population of DCs has 0 CFU/swab.

The tumor cells used in the invention are multiple myeloma cells. The multiple myeloma cells are obtained from a patient having multiple myeloma.

The tumor cells must have sufficient viability prior to fusion. The viability of the tumor cells is at least 50%, at least 60%, at least 70%, at least 80% or greater.

Prior to fusion the population of tumor cells are free of components used during the production, e.g., cell culture components and substantially free of mycoplasm, endotoxin, and microbial contamination. Preferably, the population of tumor cell population has less than 10, 5, 3, 2, or 1 CFU/swab. Most preferably the population of tumor cells has 0 CFU/swab. The endotoxin level in the population of tumor cells is less than 20 EU/mL, less than 10 EU/mL or less than 5 EU/mL.

If the tumor cells die or at least fail to proliferate in the presence of a given reagent and this sensitivity can be overcome by the fusion with DCs, the post-fusion cell mixtures containing the fused as well as the parental cells may optionally be incubated in a medium containing this reagent for a period of time sufficient to eliminate most of the unfused cells. The fusion product is used directly after the fusion process (e.g., in antigen discovery screening methods or in therapeutic methods) or after a short culture period.

Fused cells are irradiated prior to clinical use.

In the event that the fused cells lose certain DC characteristics such as expression of the APC-specific T-cell stimulating molecules, primary fused cells can be refused with dendritic cells to restore the DC phenotype. The refused cells (i.e., secondary fused cells) are found to be highly potent APCs. The fused cells can be refused with the dendritic or non-dendritic parental cells as many times as desired.

Fused cells that express MHC class II molecules, B7, or other desired T-cell stimulating molecules can also be selected by panning or fluorescence-activated cell sorting with antibodies against these molecules.

Fusion between the DCs and the tumor cells can be carried out with well-known methods such as those using polyethylene glycol (“PEG”), Sendai virus, or electrofusion. DCs are autologous or allogeneic. (See, e.g., U.S. Pat. No. 6,653,848, which is herein incorporated by reference in its entirety). The ratio of DCs to tumor cells in fusion can vary from 1:100 to 1000:1, with a ratio higher than 1:1 being preferred. Preferably, the ratio is 1:1, 5:1, or 10:1. Most preferably, the ratio of DCs to tumor cells is 10:1 or 3:1. After fusion, unfused DCs usually die off in a few days in culture, and the fused cells can be separated from the unfused parental non-dendritic cells by the following two methods, both of which yield fused cells of approximately 50% or higher purity, i.e., the fused cell preparations contain less than 50%, and often less than 30%, unfused cells.

Specifically, one method of separating unfused cells from fused cells is based on the different adherence properties between the fused cells and the non-dendritic parental cells It has been found that the fused cells are generally lightly adherent to tissue culture containers. Thus, if the non-dendritic parental cells are much more adherent, e.g., in the case of carcinoma cells, the post-fusion cell mixtures can be cultured in an appropriate medium for a short period of time (e.g., 5-10 days). Subsequently, the fused cells can be gently dislodged and aspirated off, while the unfused cells grow firmly attached to the tissue culture containers. Conversely, if the tumor cells grow in suspension, after the culture period, they can be gently aspirated off while leaving the fused cells loosely attached to the containers. Alternatively, the fusions are used directly without an in vitro cell culturing step.

Fused cells obtained by the above-described methods typically retain the phenotypic characteristics of DCs. For instance, these fused cells express T-cell stimulating molecules such as MHC class II protein, B7-1, B7-2, and adhesion molecules characteristic of APCs such as ICAM-1. The fused cells also continue to express cell-surface antigens of the tumor cells such as MUC1, NY-ESO, CD38 and CD138 and are therefore useful for inducing immunity against the cell-surface antigens.

In the event that the fused cells lose certain DC characteristics such as expression of the APC-specific T-cell stimulating molecules, they (i.e., primary fused cells) can be re-fused with dendritic cells to restore the DC phenotype. The re-fused cells (i.e., secondary fused cells) are found to be highly potent APCs, and in some cases, have even less tumorigenicity than primary fused cells. The fused cells can be re-fused with the dendritic or non-dendritic parental cells as many times as desired.

The phenotypic characteristics of DC/MM fusions are examined. Specifically, fusion of DCs/MM fusions co-express: CD11c, CD38, CD138, MUC-1, HLA DR, CD80, CD86, and CD83.

The fused cells may be frozen before administration. The fused cells are frozen in a solution containing 10% DMSO in 90% autologous heat inactivated autologous plasma.

The fused cells of the invention can be used to stimulate the immune system of a mammal for treatment or prophylaxis of multiple myeloma. For instance, to treat multiple myeloma in a human, a composition containing fused cells formed by his own DCs and tumor cells can be administered to him, e.g., at a site near the lymphoid tissue. In some embodiments the subject has received an autologous stem cell transplant. Preferably the fused cells are administered 30-100 days after receiving the autologous stem cell transplant. More preferably, the fused cells are administered within 4 weeks of hematopoietic recovery after the autologous stem cell transplant. Methods of determining hematopoietic recovery are well known in the art.

Alternatively, the fused cells may be administered during the early period of lymphopoietic recovery in which levels of circulating and bone marrow regulatory T cells are at a minimum or in combination with agents the target regulatory T cells. Another criteria for administering the fused cells post stem cell transplant is at a time post-transplant in which there is expansion of myeloma specific T cells as measured by the percentage of CD4 and/or CD8 T cells that express IFNγ in response to ex vivo exposure to autologous tumor lysate or the percentage of T cells that bind to tetramers or pentamers expressing myeloma specific antigens such as WT1, Survivin, NY-ESO, MUC1, and PRAME.

Preferably, the vaccine is administered to four different sites near lymphoid tissue. The composition may be given multiple times (e.g., two to five, preferably three) at an appropriate intervals, preferably, four weeks and dosage (e.g., approximately 10⁵-10⁸, e.g., about 0.5×10⁶ to 1×10⁶, fused cells per administration). Preferably each dosage contains approximately 1×10⁵ to 1×10⁶ fused cells. In addition the fused cells the patient further receives GM-CSF. The GM-CSF is administered on the day the fused cells are administered and for daily for three subsequent days. The GM-CSF is administered subcutaneously at a dose of 100 ug. The GM-CSF is administered at the site where the fused cells are administered.

Optionally, the patient further receives a checkpoint inhibitor. The check point inhibitor is administered contemporaneously with the fused cell, prior to administration of the fused cells or after administration of the fused cells. For example, the checkpoint inhibitor is administered 1 week prior to the fused cells. Preferably, the checkpoint inhibitor is administered 1 week after the fused cells. The checkpoint inhibitor is administered at 1, 2, 3, 4, 5, 6 week intervals.

By checkpoint inhibitor it is meant that at the compound inhibits a protein in the checkpoint signally pathway. Proteins in the checkpoint signally pathway include for example, PD-1, PD-L1, PD-L2, LAG3, TIM3, and CTLA-4. Checkpoint inhibitor are known in the art. For example, the checkpoint inhibitor can be a small molecule. A “small molecule” as used herein, is meant to refer to a composition that has a molecular weight in the range of less than about 5 kD to 50 daltons, for example less than about 4 kD, less than about 3.5 kD, less than about 3 kD, less than about 2.5 kD, less than about 2 kD, less than about 1.5 kD, less than about 1 kD, less than 750 daltons, less than 500 daltons, less than about 450 daltons, less than about 400 daltons, less than about 350 daltons, less than 300 daltons, less than 250 daltons, less than about 200 daltons, less than about 150 daltons, less than about 100 daltons. Small molecules can be, e.g., nucleic acids, peptides, polypeptides, peptidomimetics, carbohydrates, lipids or other organic or inorganic molecules.

Alternatively the checkpoint inhibitor is an antibody is an antibody or fragment thereof. For example, the antibody or fragment thereof is specific to a protein in the checkpoint signaling pathway, such as PD-1, PD-L1, PD-L2, LAG3, TIM3, or CTLA-4. Preferably, the checkpoint inhibitor is an antibody specific for PD-1, PD-L1, PD-L2, LAG3, TIM3, or CTLA-4.

Optionally, the patient may receive concurrent treatment with an immunomodulatory agent. These agents include lenalidomide, pomalinomide or apremilast. Lenalidomide has been shown to boost response to vaccination targeting infectious diseases and in pre-clinical studies enhances T cell response to the fusion vaccine.

Optionally the patient may undergo vaccination in combination with strategies to reduce levels of regulatory T cells. These strategies may include combining vaccination with chemotherapy, during the period of lymphopoietic reconstitution following autologous or allogeneic transplantation, and with antibodies or drugs that target regulatory T cells.

To monitor the effect of vaccination, cytotoxic T lymphocytes obtained from the treated individual can be tested for their potency against cancer cells in cytotoxic assays. Multiple boosts may be needed to enhance the potency of the cytotoxic T lymphocytes.

Compositions containing the appropriate fused cells are administered to an individual (e.g., a human) in a regimen determined as appropriate by a person skilled in the art. For example, the composition may be given multiple times (e.g., three to five times, preferably three) at an appropriate interval (e.g., every four weeks) and dosage (e.g., approximately 10⁵-10⁸, preferably about 1×10⁵ to 1×10⁶.

The composition of fused cells prior to administration to the patient must have sufficient viability. The viability of the fused cells at the time of administration is at least 50%, at least 60%, at least 70%, at least 80% or greater.

Prior to administration, the population of fused cells are free of components used during the production, e.g., cell culture components and substantially free of mycoplasm, endotoxin, and microbial contamination. Preferably, the population of fused cells has less than 10, 5, 3, 2, or 1 CFU/swab. Most preferably the population of tumor cells has 0 CFU/swab. For example, the results of the sterility testing is “negative” or “no growth”. The endotoxin level in the population of tumor cells is less than 20 EU/mL, less than 10 EU/mL or less than 5 EU/mL. The results of the myoplasm testing is “negative”.

Prior to administration, the fused cell must express at least 40%, at least 50%, at least60% CD86 as determined by immunological staining. Preferably the fused cells express at least 50% CD86.

More specifically, all final cell product must conform with rigid requirements imposed by the Federal Drug Administration (FDA). The FDA requires that all final cell products must minimize “extraneous” proteins known to be capable of producing allergenic effects in human subjects as well as minimize contamination risks. Moreover, the FDA expects a minimum cell viability of 70%, and any process should consistently exceed this minimum requirement.

### Definitions

The practice of the present invention employs, unless otherwise indicated, conventional techniques of molecular biology, microbiology, cell biology and recombinant DNA, which are within the skill of the art. See, e.g., Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel et al. eds., (1987)); the series METHODS IN ENZYMOLOGY (Academic Press, Inc.): PCR 2: A PRACTICAL APPROACH (Mi. MacPherson, B. D. Hames and G. R. Taylor eds. (1995)) and ANIMAL CELL CULTURE (Rd. Freshney, ed. (1987)).

As used herein, certain terms have the following defined meanings. As used in the specification and claims, the singular form “a”, “an” and “the” include plural references unless the context clearly dictates otherwise. For example, the term “a cell” includes a plurality of cells, including mixtures thereof.

The term “immune effector cells” refers to cells that specifically recognize an antigen present, for example on a neoplastic or tumor cell. For the purposes of this invention, immune effector cells include, but are not limited to, B cells; monocytes; macrophages; NK cells; and T cells such as cytotoxic T lymphocytes (CTLs), for example CTL lines, CTL clones, and CTLs from tumor, inflammatory sites or other infiltrates. “T-lymphocytes” denotes lymphocytes that are phenotypically CD3+, typically detected using an anti-CD3 monoclonal antibody in combination with a suitable labeling technique. The T-lymphocytes of this invention are also generally positive for CD4, CD8, or both. The term “naïve” immune effector cells refers to immune effector cells that have not encountered antigen and is intended to by synonymous with unprimed and virgin. “Educated” refers to immune effector cells that have interacted with an antigen such that they differentiate into an antigen-specific cell.

The terms “antigen presenting cells” or “APCs” includes both intact, whole cells as well as other molecules which are capable of inducing the presentation of one or more antigens, preferably with class I MHC molecules. Examples of suitable APCs are discussed in detail below and include, but are not limited to, whole cells such as macrophages, dendritic cells, B cells; purified MHC class I molecules complexed to β2-microglobulin; and foster antigen presenting cells.

Please provide some updated references for material below) Dendritic cells (DCs) are potent APCs. DCs are minor constituents of various immune organs such as spleen, thymus, lymph node, epidermis, and peripheral blood. For instance, DCs represent merely about 1% of crude spleen (see Steinman et al. (1979) J. Exp. Med 149: 1) or epidermal cell suspensions (see Schuler et al. (1985) J. Exp. Med 161:526; Romani et al. J. Invest. Dermatol (1989) 93: 600) and 0.1-1% of mononuclear cells in peripheral blood (see Freudenthal et al. Proc. Natl Acad Sci USA (1990) 87: 7698). Methods for isolating DCs from peripheral blood or bone marrow progenitors are known in the art. (See Inaba et al. (1992) J. Exp. Med 175:1157; Inaba et al. (1992) J. Exp, Med 176: 1693-1702; Romani et al. (1994) J. Exp. Med. 180: 83-93; Sallusto et al. (1994) J. Exp. Med 179: 1109-1118)). Preferred methods for isolation and culturing of DCs are described in Bender et al. (1996) J. Immun Meth. 196:121-135 and Romani et al. (1996) J. Immun. Meth 196:137-151.

Dendritic cells (DCs) represent a complex network of antigen presenting cells that arc primarily responsible for initiation of primary immunity and the modulation of immune response. (See Avigan, Blood Rev. 13:51-64 (1999); Banchereau et al., Nature 392:245-52 (1998)). Partially mature DCs are located at sites of antigen capture, excel at the internalization and processing of exogenous antigens but are poor stimulators of T cell responses. Presentation of antigen by immature DCs may induce T cell tolerance. (See Dhodapkar et al., J Exp Med. 193:233-38 (2001)). Upon activation, DCs undergo maturation characterized by the increased expression of costimulatory molecules and CCR7, the chemokine receptor which promotes migration to sites of T cell traffic in the draining lymph nodes. Tumor or cancer cells inhibit DC development through the secretion of 1L-10, TGF-β, and VEGF resulting in the accumulation of immature DCs in the tumor bed that potentially suppress anti-tumor responses. (See Allavena et al., Eur. J. Immunol. 28:359-69 (1998); Gabrilovich et al., Clin Cancer Res. 3:483-90 (1997); Gabrilovich et al., Blood 92:4150-66 (1998); Gabrilovich, Nat Rev Immunol 4:941-52 (2004)). Conversely, activated DCs can be generated by cytokine mediated differentiation of DC progenitors ex vivo. DC maturation and function can be further enhanced by exposure to the toll like receptor 9 agonist, CPG ODN. Moreover, DCs can be manipulated to present tumor antigens potently stimulate anti-tumor immunity. (See Asavaroenhchai et al., Proc Natl Acad Sci USA 99:931-36 (2002); Ashley et al., J Exp Med 186:1177-82 (1997)).

“Foster antigen presenting cells” refers to any modified or naturally occurring cells (wild-type or mutant) with antigen presenting capability that are utilized in lieu of antigen presenting cells (“APC”) that normally contact the immune effector cells they are to react with. In other words, they are any functional APCs that T cells would not normally encounter in vivo.

It has been shown that DCs provide all the signals required for T cell activation and proliferation. These signals can be categorized into two types. The first type, which gives specificity to the immune response, is mediated through interaction between the T-cell receptor/CD3 (“TCR/CD3”) complex and an antigenic peptide presented by a major histocompatibility complex (“MHC”) class I or II protein on the surface of APCs. This interaction is necessary, but not sufficient, for T cell activation to occur. In fact, without the second type of signals, the first type of signals can result in T cell anergy. The second type of signals, called costimulatory signals, are neither antigen-specific nor MHC restricted, and can lead to a full proliferation response of T cells and induction of T cell effector functions in the presence of the first type of signals.

Thus, the term “cytokine” refers to any of the numerous factors that exert a variety of effects on cells, for example, inducing growth or proliferation. Non-limiting examples of cytokines include, IL-2, stem cell factor (SCF), IL-3, IL-6, IL-7, IL-12, IL-15, G-CSF, GM-CSF, IL-1α, IL-1β, MIP-1α, LIF, c-kit ligand, TPO, and flt3 ligand. Cytokines are commercially available from several vendors such as, for example, Genzyme Corp. (Framingham, Mass.), Genentech (South San Francisco, Calif.), Amgen (Thousand Oaks, Calif.) and Immunex (Seattle, Wash.). It is intended, although not always explicitly stated, that molecules having similar biological activity as wild-type or purified cytokines (e.g., recombinantly produced cytokines) are intended to be used within the spirit and scope of the invention and therefore are substitutes for wild-type or purified cytokines.

“Costimulatory molecules” are involved in the interaction between receptor-ligand pairs expressed on the surface of antigen presenting cells and T cells. One exemplary receptor-ligand pair is the B7 co-stimulatory molecules on the surface of DCs and its counter-receptor CD28 or CTLA-4 on T cells. (See Freeman et al. (1993) Science 262:909-911; Young et al. (1992) J. Clin. Invest 90: 229; Nabavi et al. Nature 360:266)). Other important costimulatory molecules include, for example, CD40, CD54, CD80, and CD86. These are commercially available from vendors identified above.

A “hybrid” cell refers to a cell having both antigen presenting capability and also expresses one or more specific antigens. In one embodiment, these hybrid cells are formed by fusing, in vitro, APCs with cells that are known to express the one or more antigens of interest. As used herein, the term “hybrid” cell and “fusion” cell are used interchangeably.

A “control” cell refers to a cell that does not express the same antigens as the population of antigen-expressing cells.

The term “culturing” refers to the in vitro propagation of cells or organisms on or in media of various kinds, it is understood that the descendants 30 of a cell grown in culture may not be completely identical (L e., morphologically, genetically, or phenotypically) to the parent cell. By “expanded” is meant any proliferation or division of cells.

An “effective amount” is an amount sufficient to effect beneficial or desired results. An effective amount can be administered in one or more administrations, applications or dosages. For purposes of this invention, an effective amount of hybrid cells is that amount which promotes expansion of the antigenic-specific immune effector cells, e.g., T cells.

An “isolated” population of cells is “substantially free” of cells and materials with which it is associated in nature. By “substantially free” or “substantially pure” is meant at least 50% of the population are the desired cell type, preferably at least 70%, more preferably at least 80%, and even more preferably at least 90%. An “enriched” population of cells is at least 5% fused cells. Preferably, the enriched population contains at least 10%, more preferably at least 20%, and most preferably at least 25% fused cells.

The term “autogeneic”, or “autologous”, as used herein, indicates the origin of a cell. Thus, a cell being administered to an individual (the “recipient”) is autogeneic if the cell was derived from that individual (the “donor”) or a genetically identical individual (i.e., an identical twin of the individual). An autogeneic cell can also be a progeny of an autogeneic cell. The term also indicates that cells of different cell types are derived from the same donor or genetically identical donors. Thus, an effector cell and an antigen presenting cell are said to be autogeneic if they were derived from the same donor or from an individual genetically identical to the donor, or if they are progeny of cells derived from the same donor or from an individual genetically identical to the donor.

Similarly, the term “allogeneic”, as used herein, indicates the origin of a cell. Thus, a cell being administered to an individual (the “recipient”) is allogeneic if the cell was derived from an individual not genetically identical to the recipient. In particular, the term relates to non-identity in expressed MHC molecules. An allogeneic cell can also be a progeny of an allogeneic cell. The term also indicates that cells of different cell types are derived from genetically nonidentical donors, or if they are progeny of cells derived from genetically non-identical donors. For example, an APC is said to be allogeneic to an effector cell if they are derived from genetically non-identical donors.

A “subject” is a vertebrate, preferably a mammal, more preferably a human. Mammals include, but are not limited to, murines, simians, humans, farm animals, sport animals, and pets.

As used herein, “genetic modification” refers to any addition, deletion or disruption to a cell's endogenous nucleotides.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors and the like. In aspects where gene transfer is mediated by a retroviral vector, a vector construct refers to the polynucleotide comprising the retroviral genome or part thereof, and a therapeutic gene.

As used herein, the terms “retroviral mediated gene transfer” or “retroviral transduction” carries the same meaning and refers to the process by which a gene or a nucleic acid sequence is stably transferred into the host cell by virtue of the virus entering the cell and integrating its genome into the host cell genome. The virus can enter the host cell via its normal mechanism of infection or be modified such that it binds to a different host cell surface receptor or ligand to enter the cell.

Retroviruses carry their genetic information in the form of RNA. However, once the virus infects a cell, the RNA is reverse-transcribed into the DNA form that integrates into the genomic DNA of the infected cell. The integrated DNA form is called a provirus.

In aspects where gene transfer is mediated by a DNA viral vector, such as a adenovirus (Ad) or adeno-associated virus (AAV), a vector construct refers to the polynucleotide comprising the viral genome or part thereof, and a therapeutic gene. Adenoviruses (Ads) are a relatively well characterized, homogenous group of viruses, including over 50 serotypes. (See, e.g., WO 95/27071). Ads are easy to grow and do not integrate into the host cell genome. Recombinant Ad-derived vectors, particularly those that reduce the potential for recombination and generation of wild-type virus, have also been constructed. (See, WO 95/00655; WO 95/11984). Wild-type AAV has high infectivity and specificity integrating into the host cells genome. (See Hermonat and Muzyczka (1984) PNAS USA 81:6466-6470; Lebkowski et al., (1988) Mol Cell Biol 8:3988-3996).

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression. Examples of suitable vectors are viruses, such as baculovirus and retrovirus, bacteriophage, cosmid, plasmid, fungal vectors and other recombination vehicles typically used in the art which have been described for expression in a variety of eucaryotie and prokaryotic hosts, and may be used for gene therapy as well as for simple protein expression.

Among these are several non-viral vectors, including DNA/liposome complexes, and targeted viral protein DNA complexes. To enhance delivery to a cell, the nucleic acid or proteins of this invention can be conjugated to antibodies or binding fragments thereof which bind cell surface antigens, e.g., TCR, CD3 or CD4. Liposomes that also comprise a targeting antibody or fragment thereof can be used in the methods of this invention. This invention also provides the targeting complexes for use in the methods disclosed herein.

Polynucleotides are inserted into vector genomes using methods well known in the art. For example, insert and vector DNA can be contacted, under suitable conditions, with a restriction enzyme to create complementary ends on each molecule that can pair with each other and be joined together with a ligase. Alternatively, synthetic nucleic acid linkers can be ligated to the termini of restricted polynucleotide. These synthetic linkers contain nucleic acid sequences that correspond to a particular restriction site in the vector DNA. Additionally, an oligonucleotide containing a termination codon and an appropriate restriction site can be ligated for insertion into a vector containing, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV4O for mRNA stability; SV40 polyoma origins of replication and ColEI for proper episomal replication; versatile multiple cloning sites; and T7 and SP6 RNA promoters for in vitro transcription of sense and antisense RNA. Other means are well known and available in the art.

As used herein, “expression” refers to the process by which polynucleotides are transcribed into mRNA and translated into peptides, polypeptides, or proteins. If the polynucleotide is derived from genomic DNA, expression may include splicing of the mRNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG (Sambrook et al. (1989), supra). Similarly, a eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods well known in the art, for example, the methods described above for constructing vectors in general.

The terms “major histocompatibility complex” or “MHC” refers to a complex of genes encoding cell-surface molecules that are required for antigen presentation to immune effector cells such as T cells and for rapid graft rejection. In humans, the MHC complex is also known as the HLA complex. The proteins encoded by the MHC complex are known as “MHC molecules” and are classified into class I and class II MHC molecules. Class I MHC molecules include membrane heterodimeric proteins made up of an α chain encoded in the MHC associated noncovalently with β2-microglobulin. Class I MHC molecules are expressed by nearly all nucleated cells and have been shown to function in antigen presentation to CD8+ T cells. Class I molecules include HLA-A, -B, and -C in humans. Class TI MHC molecules also include membrane heterodimeric proteins consisting of noncovalently associated and J3 chains. Class II MHCs are known to function in CD4+ T cells and, in humans, include HLA-DP, -DQ, and DR. The term “MHC restriction” refers to a characteristic of T cells that permits them to recognize antigen only after it is processed and the resulting antigenic peptides are displayed in association with either a class T or class II MHC molecule. Methods of identifying and comparing MHC are well known in the art and are described in Allen M. et al. (1994) Human Imm. 40:25-32; Santamaria P. et al. (1993) Human Imm. 37:39-50; and Hurley C. K. et al. (1997) Tissue Antigens 50:401-415.

The term “sequence motif” refers to a pattern present in a group of 15 molecules (e.g., amino acids or nucleotides). For instance, in one embodiment, the present invention provides for identification of a sequence motif among peptides present in an antigen. In this embodiment, a typical pattern may be identified by characteristic amino acid residues, such as hydrophobic, hydrophilic, basic, acidic, and the like.

The term “peptide” is used in its broadest sense to refer to a compound of two or more subunit amino acids, amino acid analogs, or peptidomimetics. The subunits may be linked by peptide bonds. In another embodiment, the subunit may be linked by other bonds, e.g. ester, ether, etc.

As used herein the term “amino acid” refers to either natural and/or 25 unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

As used herein, “solid phase support” is used as an example of a “carrier” and is not limited to a specific type of support. Rather a large number of supports are available and are known to one of ordinary skill in the art. Solid phase supports include silica gels, resins, derivatized plastic films, glass beads, cotton, plastic beads, alumina gels. A suitable solid phase support may be selected on the basis of desired end use and suitability for various synthetic protocols. For example, for peptide synthesis, solid phase support may refer to resins such as polystyrene (e.g., PAM-resin obtained from Bachem Inc., Peninsula Laboratories, etc.), POLYHIPE® resin (obtained from Aminotech, Canada), polyamide resin (obtained from Peninsula Laboratories), polystyrene resin grafted with polyethylene glycol (TentaGel®, Rapp Polymere, Tubingen, Germany) or polydimethylacrylamide resin (obtained from MilligenlBiosearch, California). In a preferred embodiment for peptide synthesis, solid phase support refers to polydimethylacrylamide resin.

The term “aberrantly expressed” refers to polynucleotide sequences in a cell or tissue which are differentially expressed (either over-expressed or under-expressed) when compared to a different cell or tissue whether or not of the same tissue type, i.e., lung tissue versus lung cancer tissue.

“Host cell” or “recipient cell” is intended to include any individual cell or cell culture which can be or have been recipients for vectors or the incorporation of exogenous nucleic acid molecules, polynucleotides and/or proteins. It also is intended to include progeny of a single cell, and the progeny may not necessarily be completely identical (in morphology or in genomic or total DNA complement) to the original parent cell due to natural, accidental, or deliberate mutation. The cells may be prokaryotic or eukaryotic, and include but are not limited to bacterial cells, yeast cells, animal cells, and mammalian cells, e.g., murine, rat, simian or human.

An “antibody” is an immunoglobulin molecule capable of binding an antigen. As used herein, the term encompasses not only intact immunoglobulin molecules, but also anti-idiotypic antibodies, mutants, fragments, fusion proteins, humanized proteins and modifications of the immunoglobulin molecule that comprise an antigen recognition site of the required specificity.

An “antibody complex” is the combination of antibody and its binding partner or ligand.

A “native antigen” is a polypeptide, protein or a fragment containing an epitope, which induces an immune response in the subject.

The term “isolated” means separated from constituents, cellular and otherwise, in which the polynucleotide, peptide, polypeptide, protein, antibody, or fragments thereof, are normally associated with in nature. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragments thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as glycosylation pattern. Although not explicitly stated for each of the inventions disclosed herein, it is to be understood that all of the above embodiments for each of the compositions disclosed below and under the appropriate conditions, are provided by this invention. Thus, a non-naturally occurring polynucleotide is provided as a separate embodiment from the isolated naturally occurring polynucleotide. A protein produced in a bacterial cell is provided as a separate embodiment from the naturally occurring protein isolated from a eucaryotic cell in which it is produced in nature.

A “composition” is intended to mean a combination of active agent and another compound or composition, inert (for example, a detectable agent, carrier, solid support or label) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON′S PHARM. SCI, 15th Ed. (Mack Publ. Co., Easton (1975)).

As used herein, the term “inducing an immune response in a subject” is a term well understood in the art and intends that an increase of at least about 2-fold, more preferably at least about 5-fold, more preferably at least about 10-fold, more preferably at least about 100-fold, even more preferably at least about 500-fold, even more preferably at least about 1000-fold or more in an immune response to an antigen (or epitope) can be detected (measured), after introducing the antigen (or epitope) into the subject, relative to the immune response (if any) before introduction of the antigen (or epitope) into the subject. An immune response to an antigen (or epitope), includes, but is not limited to, production of an antigen-specific (or epitope-specific) antibody, and production of an immune cell expressing on its surface a molecule which specifically binds to an antigen (or epitope). Methods of determining whether an immune response to a given antigen (or epitope) has been induced are well known in the art. For example, antigen specific antibody can be detected using any of a variety of immunoassays known in the art, including, but not limited to, ELISA, wherein, for example, binding of an antibody in a sample to an immobilized antigen (or epitope) is detected with a detectably-labeled second antibody (e.g., enzyme-labeled mouse anti-human Ig antibody) Immune effector cells specific for the antigen can be detected any of a variety of assays known to those skilled in the art, including, but not limited to, FACS, or, in the case of CTLs, ⁵¹CR-release assays, or ³H-thymidine uptake assays.

By substantially free of endotoxin is meant that there is less endotoxin per dose of cell fusions than is allowed by the FDA for a biologic, which is a total endotoxin of 5 EU/kg body weight per day.

By substantially free for mycoplasma and microbial contamination is meant as negative readings for the generally accepted tests know to those skilled in the art. For example, mycoplasm contamination is determined by subculturing a cell sample in broth medium and distributed over agar plates on day 1, 3, 7, and 14 at 37° C. with appropriate positive and negative controls. The product sample appearance is compared microscopically, at 100×, to that of the positive and negative control. Additionally, inoculation of an indicator cell culture is incubated for 3 and 5 days and examined at 600× for the presence of mycoplasmas by epifluorescence microscopy using a DNA-binding fluorochrome. The product is considered satisfactory if the agar and/or the broth media procedure and the indicator cell culture procedure show no evidence of mycoplasma contamination.

The sterility test to establish that the product is free of microbial contamination is based on the U.S. Pharmacopedia Direct Transfer Method. This procedure requires that a pre-harvest medium effluent and a pre-concentrated sample be inoculated into a tube containing tryptic soy broth media and fluid thioglycollate media. These tubes are observed periodically for a cloudy appearance (turpidity) for a 14 day incubation. A cloudy appearance on any day in either medium indicate contamination, with a clear appearance (no growth) testing substantially free of contamination.

### EXAMPLES

### Example 1

### Clinical Study Design for Vaccination of Patients with Multiple Myeloma Undergoing Autologous Hematopoeitic Stem Cell Transplantation with Dendritic Cell Tumor Fusions

The primary objective of the study is: To assess the toxicity associated with vaccination of multiple myeloma patients with dendritic cell/myeloma fusions and GM-CSF prior to stem cell mobilization and following high dose chemotherapy with stem cell rescue. The secondary objectives of the study are: To determine whether tumor specific cellular and humoral immunity can be induced by serial vaccination with DC/tumor cell fusions in conjunction with high dose chemotherapy with stem cell rescue. To explore the relationship between immune recovery post-transplant, vaccine characteristics and response to vaccination. To determine if vaccination with DC/tumor cell fusions results in clinical disease response in patients with evidence of residual disease post-transplant To determine the time to disease progression for patients undergoing high dose chemotherapy in conjunction with fusion cell vaccination.

### Inclusion Criteria:

1. Patients with multiple myeloma who are potential candidates for high dose chemotherapy with stem cell rescue

2. Patients with measurable disease as defined by a history of an elevated M component in plasma, urine, or free kappa/lambda light chains in the serum

3. Patients must be ≧18 years old.

4. Patients must have ECOG performance status of 0-1 with a greater than nine week life expectancy.

5. Patients with ≧20% bone marrow involvement or plasmacytoma amenable to resection under local anesthesia

6. Women of childbearing age must have a negative pregnancy test, and adequate contraception method(s) must be documented.

7. DLCO (adjusted) >50%

8. Cardiac Ejection Fraction >45%

9. Laboratories:

WBC ≧2.0×103/uL

Bilirubin ≦2.0 mg/dL AST/ALT <3× ULN Creatinine ≦2.0 mg/dL

### Exclusion Criteria:

1. Patients with a history of clinically significant venous thromboembolism will be excluded. Patients without a history of prior thrombus who develop a thrombotic event while on thalidomide will be considered on a case by case basis.

2. Patients must not have clinically significant autoimmune disease.

3. Because of compromised cellular immunity and limited capacity to respond to vaccination, patients who are HIV+ will be excluded.

4. Patients must not have serious intercurrent illness such as infection requiring IV antibiotics, or significant cardiac disease characterized by significant arrhythmia, ischemic coronary disease or congestive heart failure

5. Pregnant and lactating women will be excluded; all premenopausal patients will undergo pregnancy testing. Men will agree to not father a child while on protocol treatment. Men and women will practice effective birth control while receiving protocol treatment.

### Eligibility Prior to Pre-Transplant Fusion Vaccination and Mobilization

1. Patients without evidence of disease progression following most recent pre-transplant therapy. (Patient may receive a maximum of 1 year of therapy prior to pre-transplant vaccination)

2. Pre-transplant vaccination to be initiated at least 4 weeks and not more than 8 weeks since last chemotherapy and at least 2 weeks and not more than 8 weeks since last biological therapy (i.e. steroids, thalidomide, velcade)

3. Patients without evidence of ongoing grade III-IV toxicity related to pre-transplant therapy

4. Patient eligible for high dose chemotherapy as determined by lack of significant organ toxicity or serious intercurrent medical illness as defined above, and institutional criteria including:

Platelets >50,000/uL WBC >2.0×103/uL DLCO >50% predicted

Cardiac Ejection Fraction >45% Serum total bilirubin <2.0 mg/dL AST/ALT <3.0× ULN Serum creatinine <2.0 mg/dL ECOG performance status of 0-1

### Eligibility Prior to High Dose Chemotherapy

1. Minimum of 2×10⁶ CD34+ cells/kg collected at mobilization

2. Patients without evidence of ongoing grade III-IV toxicity related to mobilization therapy

Eligibility Prior to Post-Transplant Vaccination with Fusion Cells

1. Resolution of all transplant related grade III-IV toxicity

2. Laboratories: WBC >2.0×10³/uL Platelets >50,000/uL Bilirubin <2.0 mg/dL Creatinine <2.0 mg/dL AST/ALT <3.0× ULN

### Baseline/Enrollment Testing

### Within 21 Days of Registration

1—Medical History, Physical Exam, Assessment of performance status

2—Bone Marrow Aspirate/biopsy

3—Skeletal Survey and/or other appropriate radiological assessment of the disease status

4—Ejection Fraction

5—Pulmonary function tests

6—Electrocardiogram (EKG)

7—Laboratory evaluation:

Serum Protein Electropheresis(SPEP), 24 hour urine quantitative protein and electropheresis (24 hour UPEP) (when appropriate), or free kappa lambda light chain quantative immunoglobulins(IgG, IgA, IgM), β2 microglobulin, Pregnancy Test if applicable, TSH, Erythrocyte Sedimentation Rate (ESR), Antinuclear Antibody (ANA), HIV Test, Hepatitis B surface Ag

### Within 8 Days of Registration

CBC with differential,

Liver Function Tests (LFTs) (including; ALT, AST, total bilirubin, direct

bilirubin, LDH, Alkaline Phosphatase), Electrolytes (Na, K, Cl, CO2, Ca, Mg, PO4), BUN Creat,

### Within 8 Days of Leukapheresis Collection

The following standard assessments will be performed:

Standard Infection serologies required for storage of cellular products, PT/PTT, A,B,O and Rh blood group typing if it has not already been performed, CBC, electrolytes BUN, Creatinine, and liver function tests are repeated if greater than 14 days separate baseline/enrollment testing and leukapheresis

If tumor cells are harvested at a different time (more than 14 days from the time of leukapheresis) these tests are also obtained at that time: CBC, electrolytes BUN, Creatinine, and liver function tests. If tumor is obtained before leukapheresis then 5 green tops of plasma will be obtained to store the tumor cells and infectious serolgies* will be obtained within 7 days of the tumor collection.

### Isolation of Tumor Cells

Autologous tumor will be isolated from bone marrow specimens or a resected plasmacytoma subjected to mechanical disruption. Bone marrow aspirates will be obtained (20-30 cc) under local anesthesia and mononuclear cells will be isolated by ficoll density gradient centrifugation. Autologous plasma will be obtained during leukapheresis collections or alternatively by harvesting supernatant following ficoll centrifugation of 50-100 ml of peripheral blood. Bone marrow mononuclear cells will be cultured in media with 1% autologous plasma. An aliquot of the tumor cells will undergo immunohistochemical staining and/or FACS analysis for expression of CD138, CD38, MUC-1, class II and co-stimulatory molecules. The percentage of myeloma cells will be determined by quantifying cells that are CD138+ and/or CD38+. The percentage of myeloma cells must be ≧30% of the total population to proceed with the fusion. If the percentage of myeloma cells is <30% then the cells may be cultured for a longer interval in an effort to select for the malignant clone. A repeat marrow aspirate may be performed if the first marrow aspirate does not yield adequate tumor cells. The ability of the myeloma cells to induce proliferation of allogeneic T cells will be measured. Myeloma cells may be frozen in 10% DMSO/90% autologous plasma stored in liquid nitrogen. In this setting, myeloma cells will subsequently be thawed, recultured and viability as well as gram stain will be assessed. If sufficient numbers of myeloma cells can be obtained from the cultured material, the appropriate number of cells for a given dose level will be harvested at the time of fusion. An aliquot from this sample will undergo microbiological assessment. When cell yields allow, three doses of 1×10⁵ to 1×10⁶ cells (based upon cell availability) will be resuspended in PBS, irradiated to 6,000 rads (60 Gy) and frozen in liquid nitrogen for subsequent DTH testing. Remaining cells may be frozen for use in subsequent in vitro assays. Tumor lysate will be prepared by freeze/thawing or sonication of an aliquot of tumor cells for immunological analysis.

### Isolation of DC

Patients will undergo leukapheresis to obtain adequate numbers of PBMC. When possible, this will be performed via peripheral access. If peripheral access is inadequate, patients will undergo placement of a temporary central venous catheter. Patients with WBC <4.0×103/ul may receive 1-2 doses of GM-CSF (5 ug/kg) prior to leukapheresis to improve white blood cell yields. After completion of leukapheresis, PBMC will be quantified. If an inadequate yield of PBMC is obtained for the patient's dose requirement, a repeat procedure will be performed.

PBMC will be isolated from the leukapheresis product and cultured in the presence of autologous plasma for 1-2 hours. The non-adherent fraction, rich in T cells, will be removed. The remaining population will be cultured in the presence of 1% autologous plasma/RPMI medium with 12.5 ng/ml rhIL-4 and 1000 U/ml GM-CSF for five to seven days. 25 ng/ml of TNF will be then be added for 48-72 hours to enhance DC maturation. In some cases, aliquots of DC progenitors will be frozen in 10% DMSO/90% RPMI 1640 containing autologous plasma and stored in liquid nitrogen. The cells will subsequently be thawed and placed in culture in RPMI 1640 with GM-CSF, IL-4, and TNFα. Viability and gram stain will be assessed prior to fusion. These cells will be assessed for morphologic characteristics and expression of characteristic DC markers that include CD11c, HLA DR, CD80, CD86, and CD83. In addition expression of CD38, CD138, and MUC-1 will be determined. Functional properties will be assessed using MLR assays in which DC will be co-cultured with allogeneic T cells. T cell proliferation will be measured via tritiated thymidine incorporation.

### Preparation of DC/Tumor Fusions

Vaccine preparation may occur prior to the initiation, during, or upon completion of induction therapy. Samples will be frozen as outlined below and thawed at the time of vaccine administration. Tumor cells and DC at ratio of 1:10-1:3 (dependent on cell yields) will be mixed and extensively washed in serum-free medium (RPMI 1640). After low speed centrifugation, the cell pellets will be re-suspended in 500 μl of 50% solution of polyethylene glycol (PEG) in Dulbecco's phosphate buffered saline without Ca++, Mg++. After one to five minutes, the PEG will be progressively diluted by the slow addition of serum-free medium. The cells will be washed free of PEG and cultured in RPMI 1640 with 10% autologous plasma and GM-CSF in a 5% CO2 atmosphere at 37° C. The percentage of the cell population that represent DC/tumor fusions will be determined by quantifying the cells as defined by dual expression of unique DC and myeloma markers such as: a) CD86 and CD38 or MUC-1 or CD138; or b) CD83 and CD38 or CD138 or MUC-1; c) CD11c and CD38 or CD138 or MUC or d) if the myeloma cells do not express DR, then DR and CD38 or CD138 or MUC-1 as measured by immunocytochemical staining and/or FACS analysis. Dosing will be determined by the absolute number of fusion cells identified in this manner.

The fusion cells will then be separated into appropriate aliquots of fusion cells and frozen in 10% DMSO/90% autologous plasma in liquid nitrogen. Fusion vaccine doses containing 5×105-5×106 fusion cells will be prepared (utilizing the maximum possible dose dependent on cell yields to generate 3-4 vaccines).

### Vaccine Design

The first cohort of patients will only undergo 3 vaccinations post-transplant. Patients will not undergo vaccination if a minimum of 2 doses of the vaccine are not generated. If >2 patients of the first 6 patients or >4 patients of the total cohort (14 patients) experiences treatment limiting toxicity (as defined below), than no further patients will be enrolled. The second cohort of 14 patients will undergo pre-transplant vaccination and post- transplant boosting for a total of 4 doses (1 pre-transplant/3 post-transplant). Stopping rules as outlined for the first cohort will be followed.

If an inadequate number of fusion cells are available, 3 doses will be prepared (1 pre-transplant/2 post-transplant). Patients in the second cohort who are unable to generate 3 doses of fusion cells will not proceed with vaccination. At the appropriate time, these samples will be thawed, irradiated with 30 Gy and administered to the patient. A document outlining the staining characteristics, viability, and microbiological analyses (mycoplasma, endotoxin, and sterility) will be generated for each patient as a certificate of analysis.

Enrollment to the first cohort may continue until 14 patients have completed 1 month follow up following the final vaccine. A maximum of 28 patients will be treated in the first cohort. If <4 of the initial 14 patients experience TLT (defined below) at one month following the final vaccination, enrollment to the second cohort will begin.

### Pre-Transplant Therapy

Patients may have received a maximum of 1 year of induction therapy prior to pre-transplant vaccination or mobilization chemotherapy. The choice of pre-transplant therapy will be decided upon by the treating physician.

Pre-Transplant Vaccination with Fusion Cells

Following the completion of pre-transplant therapy, patients will be evaluated for eligibility to proceed with stem cell mobilization (cohort 1) or pre-transplant vaccination followed by stem cell mobilization (cohort 2). In cohort 2, eligible patients will be vaccinated with a single dose of fusion cells. Vaccination will be administered subcutaneously in the area of the upper thigh using a 25-gauge ⅝-inch needle. On the day of vaccination and for three days afterwards, patients will receive 100 ug of GM-CSF administered subcutaneously at the site of vaccination. Seven to 21 days following vaccination patients will proceed with mobilization chemotherapy. Mobilization chemotherapy (cohort 1) or pre-transplant vaccination (cohort 2) is to begin 4 to 8 weeks following the last chemotherapy or 2 to 8 weeks following last biological therapy (steroids, thalidomide, velcade).

Post-Transplant Vaccination with Fusion Cells

Fourteen to twenty eight days following stem cell infusion, patients will be reassessed for eligibility for post-transplant vaccination. Patients demonstrating hematopoietic engraftment and meeting eligibility criteria outlined in section 4.5 will undergo vaccination between 14-42 days post transplant. Those patients not meeting criteria by day 42 will continue to be re-assessed up to day 180 post-transplant vaccination. Those patients not meeting criteria will not proceed with vaccination. In the upper thigh region, patients will be vaccinated with fusion cells. The site will be alternated for each vaccine administration (right and left extremity). Vaccination will be administered subcutaneously using a 25-gauge ⅝-inch needle. On the day of vaccination, the clinical research nurse will administer 100 ug of GM-CSF subcutaneously at the site of DC/Fusion vaccination. The patient will be trained to inject the remaining three GM-CSF injections (100 ug dose once a day) for self-administration subcutaneously at home. Patients will undergo vaccination every 28 days (+/−2 days) for a total of 2-3 doses post-transplant (dependent on cell yields). Vaccination may be delayed if the patient experiences a clinically significant infection (grade II or higher). In this setting, vaccination may be held for up to three weeks until event has resolved to grade I or lower.

### Schedule of Testing

### Pre-Mobilization (Cohort 1)/Pre-Transplant Vaccine (Cohort 2)

### Within 29 Days of Mobilization Chemotherapy (Cohort 1) or Pre-Transplant Vaccination (Cohort 2), Patients Will Undergo

1—Medical History, Physical Exam, Assessment of performance status,

2—Laboratory evaluations: Serum Protein Electropheresis (SPEP), 24-hour urine quantitative protein and electropheresis (24 hour UPEP) free kappa lambda light chain (only in patients where this is used as a measure of disease) quantative immunoglobulins(IgG, IgA, IgM), β2 microglobulin, Pregnancy Test if applicable, Erythrocyte Sedimentation Rate (ESR), Antinuclear Antibody (ANA), T cell subsets PT/PTT, HIV-1, HIV-2 antibodies, Hepatitis B Surface Antigen, Hepatitis B core antibody; Hepatitis C antibody, A,B,O and Rh blood group typing, Bone marrow aspirate/biopsy and an additional sample of 5-10 cc maybe collected for immune monitoring studies, Assessment of ejection fraction, Pulmonary function testing, Standard BMT Infection Serologies EKG, Skeletal Survey

### Within 15 Days of Mobilization Chemotherapy (Cohort 1) or Pre-Transplant Vaccination (Cohort 2), Patients Will Undergo:

CBC with differential,

Liver Function Tests (LFTs) (including; ALT, AST, total bilirubin, direct bilirubin, LDH, Alkaline Phosphatase),

Electrolytes (Na, K, Cl, CO2, Ca, Mg, PO4), BUN Creat, TSH

Research Blood Work: DC subsets, assessment of cellular and humoral immunologic response (including tumor lysate induced T cell proliferation, IFNγ expression, tetramer response, and/or antibody studies of patient sera)

Skin tests: DTH response to irradiated tumor cells

DTH response to candida

### Pre-Transplant Vaccination (Cohort 2) +/−2 Days:

1—Medical History, Physical Exam, Assessment of Performance Status

2—Laboratory evaluations: CBC with diff,

For Patients in Cohort 2, the Following will be Performed within 2 Days of Mobilization Chemotherapy:

1—Vaccine toxicity assessment,

2—Medical History, Physical Exam, Assessment of performance status,

3—Laboratory evaluations: CBC with diff, liver function tests (LFTs) (including; ALT, AST, total bilirubin, direct bilirubin, LDH, Alkaline Phosphatase), BUN and creatinine. Na,K,Cl,CO2

### During the Mobilization Period (Beginning on Day of High Dose Cyclphosphamide) the Following Testing Will be Done Weekly:

Vaccine toxicity assessment (cohort 2) CBC with diff.

### Transplant Period Evaluation Prior to High Dose Melphalan

Within 8 Days of Admission for High Dose Melphalan the Following Testing will be Performed:

1—Medical History, Physical Exam, Assessment of Performance Status,

2—Vaccine Associated Toxicity Assessment (cohort 2)

3—laboratory evaluation: CBC with diff,liver function tests (LFTs) (including; ALT, AST, total bilirubin, direct bilirubin, LDH, Alkaline Phosphatase), Electrolytes (Na, K, Cl, CO₂, Ca, Mg, PO4) BUN, Creatinine, serum protein electropheresis(SPEP) 24 hour urine quantitative protein and electropheresis (24 hour UPEP) free kappa lambda light chain (only in patients where this is used as a measure of disease) quantative immunoglobulins(IgG, IgA, IgM) β2 microglobulin erythrocyte sedimentation rate (ESR) antinuclear antibody (ANA).

### Example 2

### Clinical Study Design to Access Vaccination of Patients with Multiple Myeloma with Dendritic Cell Tumor Fusions Combined with Pd-1 Blockade

The study will be conducted in two stages. In the first stage, a pilot study will be conducted in which patients are treated with PD-1 Antibody following autologous transplant. The primary objective of this stage is to explore immunologic responses to PD-1 BLOCKADE in the post-transplant period. The secondary objective is to assess the toxicity of treating patients with PD-1 BLOCKADE in the post-transplant setting.

In the second stage, patients will receive a combination of PD-1 BLOCKADE and DC/myeloma fusion vaccination. The primary objective is to determine if cellular immunity is induced by treatment with monoclonal antibody PD-1 BLOCKADE and DC/myeloma fusion cells in conjunction with stem cell transplant. The secondary objectives of this stage are: To assess the toxicity associated with treating multiple myeloma patients with monoclonal antibody PD-1 blockade in combination with DC/myeloma fusion vaccine following autologous transplant; To correlate levels of circulating activated and regulatory T cells with immunologic response; To define anti-tumor effects using serum markers, radiological studies, and time to disease progression.

### Inclusion Criteria:

1. Patients with multiple myeloma who are potential candidates for high dose chemotherapy with stem cell rescue

2. Patients with measurable disease as defined by a history of an elevated M component in plasma, urine, or free kappa/lambda light chains in the serum

3. Patients must be 18 years old.

4. Patients must have ECOG performance status of 0-1 with a greater than nine week life expectancy.

5. Patients with >20% bone marrow involvement or plasmacytoma amenable to resection under local anesthesia

6. Women of childbearing age must have a negative serum pregnancy test, and adequate contraception method(s) must be documented.

7. DLCO (adjusted) >50%

8. Cardiac Ejection Fraction >45%

9. Laboratories:

WBC ≧2.0×10³/uL Bilirubin ≦2.0 mg/dL

AST/ALT <3× ULN

Creatinine ≦2.0 mg/dL HIV test must be negative

### Exclusion Criteria:

1. Patients must not have active or history of clinically significant autoimmune disease, defined as requiring systemic therapy, such as Type I diabetes. Type II diabetes, vitiligo, stable hypothyroidism, and thyroid disease well controlled with thyroid replacement will not be considered exclusion criteria.

2. Because of compromised cellular immunity and limited capacity to respond to vaccination, patients who are HIV+ will be excluded.

3. Patients must not have serious intercurrent illness such as infection requiring IV antibiotics, or significant cardiac disease characterized by significant arrhythmia, ischemic coronary disease or congestive heart failure

4. Pregnant and lactating women will be excluded; all premenopausal patients will undergo pregnancy testing. Men will agree to not father a child while on protocol treatment. Men and women will practice effective birth control while receiving protocol treatment

5. History of allogeneic bone marrow/stem cell transplant

### Eligibility Prior to Mobilization Therapy

1. Resolution of grade ITT-IV toxicity associated with pre-transplant therapy

### Eligibility Prior to High Dose Chemotherapy

1. Minimum of 2×10⁶ CD34+ cells/kg collected at mobilization

2. Patients without evidence of ongoing grade toxicity related to mobilization therapy

### Eligibility Prior to Post-Transplant Immunotherapy

1. Resolution of all transplant related grade III-IV toxicity

2. Laboratories: WBC ≧2.0×10³/uL Platelets >50,000/uL Bilirubin ≦2.0 mg/dL Creatinine ≦2.0 mg/dL AST/ALT <3.0× ULN

3. Able to produce at least 2 doses of fusion vaccine (cohort 2)—To be considered evaluable. Patients who are unable to produce at least 2 doses of fusion vaccine, but otherwise meet eligibility criteria for post-transplant immunotherapy, will be treated with PD-1 BLOCKADE alone and will be replaced.

### Isolation of Tumor Cells

Autologous tumor will be isolated from bone marrow specimens or a resected plasmacytoma subjected to mechanical disruption. Bone marrow aspirates will be obtained (20-30 cc) under local anesthesia and mononuclear cells will be isolated by ficoll density gradient centrifugation (cohort 2). Autologous plasma will be obtained during leukapheresis collections or alternatively by harvesting supernatant following ficoll centrifugation of 50-100 ml of peripheral blood. Bone marrow mononuclear cells will be cultured in media with 1% autologous plasma. An aliquot of the tumor cells will undergo immunohistochemical staining and/or FACS analysis for expression of CD138, CD38, MUC-1, class II and co-stimulatory molecules. The percentage of myeloma cells will be determined by quantifying cells that are CD138+ and/or CD38+. The percentage of myeloma cells must be ≧30% of the total population to proceed with the fusion. If the percentage of myeloma cells is <30% then the cells may be cultured for a longer interval in an effort to select for the malignant clone. A repeat marrow aspirate may be performed if the first marrow aspirate does not yield adequate tumor cells. The ability of the myeloma cells to induce proliferation of allogeneic T cells will be measured. In cohort 1, 5-10 cc of bone marrow aspirate will be obtained for immunologic analyses and DTH testing. Standard infectious serologies required for storage of cellular products will be collected as per institutional practice.

Myeloma cells may be frozen in 10% DMSO/90% autologous plasma stored in liquid nitrogen. In this setting, myeloma cells will subsequently be thawed, recultured and viability as well as gram stain will be assessed. If sufficient numbers of myeloma cells can be obtained from the cultured material, the appropriate number of cells for a given dose level will be harvested at the time of fusion. An aliquot from this sample will undergo microbiological assessment. When cell yields allow, three doses of 1×10⁵ to 1×10⁶ cells (based upon cell availability) will be resuspended in PBS and frozen at −30° C. for subsequent DTH testing. Remaining cells may be frozen for use in subsequent in vitro assays. Tumor lysate will be prepared by freeze/thawing or sonication of an aliquot of tumor cells for immunological analysis.

### Isolation of Dc (Cohort 2)

Patients will undergo leukapheresis to obtain adequate numbers of PBMC. When possible, this will be performed via peripheral access. If peripheral access is inadequate, patients will undergo placement of a temporary central venous catheter. Patients with WBC <4.0×10³/ul may receive 1-2 doses of GM-CSF (5 ug/kg) prior to leukapheresis to improve white blood cell yields. After completion of leukapheresis, PBMC will be quantified. If an inadequate yield of PBMC is obtained for the patient's dose requirement, a repeat procedure will be performed.

PBMC will be isolated from the leukapheresis product and cultured in the presence of autologous plasma for 1-2 hours. The non-adherent fraction, rich in T cells, will be removed. The remaining population will be cultured in the presence of 1% autologous plasma/RPMI medium with 12.5 ng/ml rhIL-4 and 1000 U/ml GM-CSF for five to seven days. 25 ng/ml of TNFα will be then be added for 48-72 hours to enhance DC maturation. In some cases, aliquots of DC progenitors will be frozen in 10% DMSO/90% RPMI 1640 containing autologous plasma and stored in liquid nitrogen. The cells will subsequently be thawed and placed in culture in RPMI 1640 with GM-CSF, IL-4, and TNFα. Viability and gram stain will be assessed prior to fusion.

These cells will be assessed for morphologic characteristics and expression of characteristic DC markers that include CD11c, HLA DR, CD80, CD86, and CD83. In addition expression of CD38, CD138, and MUC-1 will be determined. Functional properties will be assessed using MLR assays in which DC will be co-cultured with allogeneic T cells. T cell proliferation will be measured via tritiated thymidine incorporation.

### Preparation of Dc/Tumor Fusions (Cohort 2)

Vaccine preparation may occur prior to the initiation, during, or upon completion of induction therapy. Samples will be frozen as outlined below and thawed at the time of vaccine administration. Tumor cells and DC at ratio of 1:10-1:3 (dependent on cell yields) will be mixed and extensively washed in serum-free medium (RPMI 1640). After low speed centrifugation, the cell pellets will be re-suspended in 500 μl of 50% solution of polyethylene glycol (PEG) in Dulbecco's phosphate buffered saline without Ca⁺⁺, Mg⁺⁺. After one to five minutes, the PEG will be progressively diluted by the slow addition of serum-free medium. The cells will be washed free of PEG and cultured in RPMI 1640 with 10% autologous plasma and GM-CSF in a 5% CO₂ atmosphere at 37° C. The percentage of the cell population that represent DC/tumor fusions will be determined by quantifying the cells as defined by dual expression of unique DC and myeloma markers such as: a) CD86 and CD38 or MUC-1 or CD138; or b) CD83 and CD38 or CD138 or MUC-1; c) CD11c and CD38 or CD138 or MUC or d) if the myeloma cells do not express DR, then DR and CD38 or CD138 or MUC-1 as measured by immunocytochemical staining and/or FACS analysis. Dosing will be determined by the absolute number of fusion cells identified in this manner.

The fusion cells will then be separated into appropriate aliquots of fusion cells and frozen in 10% DMSO/90% autologous plasma in liquid nitrogen. Two to three doses of 1×10⁶ to 5×10⁶ fusion cells will be cryopreserved for subsequent vaccination. An aliquot will be harvested for immunophenotypic and microbiological analysis. Fusion cells will be radiated at 30 Gy prior to administration to prevent in vivo proliferation of unfused tumor cells. A document outlining the staining characteristics, viability, and microbiological analyses (mycoplasma, endotoxin, and sterility) will be generated for each patient as a certificate of analysis.

### Post-Transplant Immunotherapy

On day 30-100 following stem cell infusion, patients will be reassessed for eligibility for post-transplant immunotherapy. Patients demonstrating hematopoietic engraftment and meeting eligibility criteria outlined in section 5 will begin immunotherapy between 30-100 days post transplant. Patients who do not meet eligibility by day 100 will come off study.

Cohort 1: Patients will receive 3 doses of of PD-1 BLOCKADE given at 6 week intervals. Patients will receive acetaminophen 500-1000 mg orally and anti-histamine (for eg. Diphenhydramine 25-50 mg) intravenously 20-60 minutes prior to PD-1 BLOCKADE infusion. The choice of oral antihistamine is at the investigator's discretion. Blood pressure, heart rate, and temperature will be measured after the administration of anti-histamine, and before the initiation of PD-1 BLOCKADE infusion. Vitals signs will be reviewed prior to administration of the study drug. PD-1 BLOCKADE will be infused over 2 hours; in cases where infusion rate is slowed due to an infusion related reaction, the overall infusion time should not exceed 10 hours.

### Schedule of Testing

### Screening Evaluation:

### Within 21 Days of Registration

1—Medical History, Physical Exam, Assessment of performance status

2—Bone Marrow Aspirate/biopsy

3—Skeletal Survey and/or other appropriate radiological assessment of the disease status

4—Ejection Fraction

5—Pulmonary function tests

6—Electrocardiogram (EKG)

7—Laboratory evaluation: CBC with differential, Liver Function Tests (LFTs) (including; ALT, AST, total bilirubin, direct bilirubin, LDH, Alkaline Phosphatase), Electrolytes (Na, K, Cl, CO₂, Ca, Mg, PO4), BUN Creat, Serum Protein Electropheresis (SPEP), 24 hour urine quantitative protein and electropheresis (24 hour UPEP) (when appropriate), or free kappa lambda light chain quantative immunoglobulins (IgG, IgA, IgM), β2 microglobulin, Serum HCG pregnancy test if applicable Pregnancy testing will be performed on all pre-menopausal women subjects as a part of study screening, Erythrocyte Sedimentation Rate (ESR) Antinuclear Antibody (ANA), TSH, Hepatitis B surface Ag, HIV test

### Within 8 Days of All Leukapheresis Collections (Cohort 2):

Standard Infection serologies required for storage of cellular products* PT/PTT, INR, A,B,O and Rh blood group typing if it has not been performed in the past, CBC, electrolytes BUN, Creatinine, and liver function tests are repeated if greater than 14 days separate baseline/enrollment testing and leukapheresis.

If tumor cells are harvested at a different time (more than 14 days from the time of leukapheresis) these tests are also obtained at that time: CBC, electrolytes BUN, Creatinine, and liver function tests. If tumor is obtained before leukapheresis then 5 green tops of plasma will be obtained to store the tumor cells and infectious serologies* will be obtained within 8 days of the tumor collection.

Evaluation Prior to Mobilization Therapy (within 8 Days of Initiation of Mobilization Therapy):

1—Medical History, Physical Exam, Assessment of performance status,

2—Laboratory evaluations: CBC with differential, liver function tests (LFTs) (including; ALT, AST, total bilirubin, direct bilirubin, LDH, Alkaline Phosphatase), BUN and creatinine. Na,K,Cl,CO2, Ca, Mg, PO4

3—Research blood work will be sent for assessment

4—DTH skin testing to candida and irradiated tumor cells.

During the Mobilization Period (Beginning on First Day of Mobilization Therapy) the Following Testing will be Done Weekly: CBC with Differential
Within 8 Days of Admission for High Dose Melphalan the Following Testing will be Performed:

1—Medical History, Physical Exam, Assessment of Performance Status,

2—Laboratory evaluation:

CBC with differential,

liver function tests (LFTs) (including; ALT, AST, total bilirubin, direct bilirubin, LDH, Alkaline Phosphatase),electrolytes(Na, K, Cl, CO₂, Ca, Mg, PO4) BUN, Creatinine, serum protein electropheresis (SPEP), 24 hour urine quantitative protein and electropheresis (24 hour UPEP), free kappa lambda light chain (only in patients where this is used as a measure of disease), quantative immunoglobulins(IgG, IgA, IgM), β2 microglobulin, erythrocyte sedimentation rate (ESR), antinuclear antibody (ANA). TSH

### Guidelines for Follow-Up During the Transplant Period are as Follows:

1—Medical History, Physical Exam, Assessment of Performance Status,

2—Laboratory evaluations: CBC, BUN, Creat, lytes are performed daily

LFT's are preformed 3 times a week

### Post Transplant Immunotherapy Period: First Dose of Post Transplant Immunotherapy to be 30-100 Days Following Transplant

### Evaluation Prior to Post-Transplant Immunotherapy

Within 15 days of post-transplant immunotherapy, patients will undergo:

1—Medical History, Physical Exam, Assessment of Performance Status,

2—Bone Marrow Aspirate and Biopsy and an additional sample of 5-10 cc may be collected for immune monitoring studies,

3—EKG

4—Laboratory evaluation: CBC with differential, liver function tests (LFTs) (including; ALT, AST, total bilirubin, direct bilirubin, LDH, Alkaline Phosphatase), Electrolytes (Na, K, Cl, CO₂, Ca, Mg, PO4) BUN, creatinine serum protein electropheresis (SPEP), 24 hour urine quantitative protein and electropheresis (24 hour UPEP), free kappa lambda light chain (only in patients where this is used as a measure of disease), quantative immunoglobulins(IgG, IgA, IgM), β2 microglobulin, erythrocyte sedimentation rate (ESR), and antinuclear antibody (ANA) T cell subsets, TSH

DTH skin testing to candida and irradiated tumor cells.

### Evaluation During Post-Transplant Immunotherapy Period

The Following Evaluation will be Performed Prior to each Subsequent Dose of PD-1 BLOCKADE (for Cohort 1) or Vaccine (for Cohort 2) (+/−2 Days):

1—Medical History, Physical Exam, Assessment of Performance

### Status

2—Vaccine Associated Toxicity Assessment (cohort 2)

3—Laboratory evaluations: CBC with differential, liver function tests (LFTs) (including; ALT, AST, total bilirubin, direct bilirubin, LDH, Alkaline Phosphatase), electrolytes (Na, K, Cl, CO₂, Ca, Mg, PO4), BUN, Creatinine serum protein electropheresis (SPEP), 24 hour urine quantitative protein and electropheresis (24 hour UPEP) free kappa lambda light chain (only in patients where this is used as a measure of disease)

quantative immunoglobulins(IgG, IgA, IgM), β2 microglobulin,
erythrocyte sedimentation rate (ESR), antinuclear antibody (ANA), TSH, ECG (for patients treated on Cohort 1), T cell subsets
The Following will be Performed Prior to each PD-1 BLOCKADE Infusion for Patients Treated on Cohort 2 (+/−2 Days):

1—Medical History, Physical Exam, Assessment of Performance

### Status

2—Vaccine Associated Toxicity Assessment

3—ECG

4—Laboratory evaluations: CBC with differential, liver function tests (LFTs) (including; ALT, AST, total bilirubin, direct bilirubin, LDH, Alkaline Phosphatase),

electrolytes(Na, K, Cl, CO₂, Ca, Mg, PO4), BUN, Creatinine

4. Research blood work will be sent for assessment

The Following will be Performed Weekly; on the Weeks that Immunotherapy is Not Administered +/−2 Days:

1—Medical History, Physical Exam, Assessment of Performance Status

2—Treatment Associated Toxicity

3—Laboratory evaluations: CBC with diff liver function tests (LFTs) (including; ALT, AST, total bilirubin, direct bilirubin, LDH, Alkaline Phosphatase), electrolytes (Na, K, Cl, CO₂, Ca, Mg, PO4), BUN, Creatinine

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A method of treating multiple myeloma in a patient comprising administering to said patient within 4 weeks of hematopoietic recovery following an autologous stem cell transplant a composition comprising a population of autologous dendritic cell/multiple myeloma cell fusions (DC/MM fusions).

2. The method of claim 1, wherein the composition comprises about 1×10⁵ to 1×10⁶ DC/MM cell fusions.

3. The method of claim 1, wherein the patient receives one dose of DC/MM fusions prior to said autologous stem cell transplant.

4. The method of claim 1, wherein the composition is administered at four week intervals.

5. The method of claim 3, wherein the subject receives at least two doses of said composition.

6. The method of claim 1, further comprising administering GM-CSF

7. The method of claim 5, wherein said GMCSF is administered daily for 3 days.

8. The method of claim 5, wherein the GM-CSF is administered at a dose of 100 ug.

9. The method of claim 4, comprising further administering GM-CSF at each dose of said DC/MM cell fusions.

10. The method of claim 1, further comprising administering said subject a checkpoint inhibitor.

11. The method of claim 10, wherein the checkpoint inhibitor is administered one week after the DC/MM fusions.

12. The method of claim 10, wherein the checkpoint inhibitor is a PD1, PDL1, PDL2, TIM3, LAG3 inhibitor.

13. The method of claim 12, wherein the checkpoint inhibitor is a PD1, PDL1, TIM3, LAG3 antibody.

14. The method of claim 1, wherein the further comprising administering an agent that target regulatory T cells

15. The method of claim 1, further comprising administering said subject an immunomodulatory agent.

16. The method of claim 15 where the immunomodulatory agent is lenalidomide or pomalinomide or apremilast.

17. The method of claim 1, further comprising administering said subject a TLR agonist, CPG ODN, polyIC, or tetanus toxoid
